(19) United States
(12) Patent Application Publication
(10) Pub. No.: US 2022/0257173 A1
(43) Pub. Date: Aug. 18, 2022

(54) EXTENDED-REALITY SKIN-CONDITION-DEVELOPMENT PREDICTION AND VISUALIZATION

(71) Applicant: Optum Technology, Inc., Eden Prairie, MN (US)

(72) Inventors: Yash Sharma, Pradesh (IN); Vivek R. Dwivedi, Noida (IN); Anshul Verma, Faridabad (IN)

(21) Appl. No.: 17/249,022

(22) Filed: Feb. 17, 2021

(51) Int. Cl. A61B 5/00; A61B 5/103; A61B 5/107; G16H 50/50; G16H 30/20; G16H 30/40; G16H 50/20; G06N 20/00; G06T 19/00

(52) U.S. Cl. CPC A61B 5/441; G06T 2210/41; A61B 5/7282; A61B 5/0077; A61B 5/7445; A61B 5/1032; A61B 5/7267; A61B 5/1077; A61B 5/7278; A61B 5/4842; G16H 50/50; G16H 30/20; G16H 30/40; G16H 50/20; G06N 20/00; G06T 19/006; A61B 2562/0219; A61B 2576/02; A61B 5/7275

(57) ABSTRACT

In general, this disclosure describes techniques for automatically predicting and visualizing a future development of a skin condition of a patient. In some examples, a computing system is configured to estimate, based on sensor data, a skin-condition type for a skin condition on an affected area of a body of a patient; determine, based on the sensor data and the estimated skin-condition type, modeling data indicative of a typical development of the skin-condition type; generate, based on the sensor data and the modeling data, a 3-dimensional (3-D) model indicative of a predicted future development of the skin condition over time; generate extended reality (XR) imagery of the affected area of the body of the patient overlaid with the 3-D model; and output the XR imagery.

### TECHNICAL FIELD

The disclosure relates to medical computing systems.

### BACKGROUND

A dermatological patient may suffer from a skin condition, such as a rash, burn, abrasion, outbreak, blemish, bruise, infection, or the like.

### SUMMARY

In general, this disclosure describes systems and techniques for automatically estimating or identifying a patient's skin-condition type, predicting a future development of the skin condition over time, and visualizing the predicted future development via extended-reality (“XR”) elements. For example, techniques disclosed herein include generating and outputting XR imagery of a predicted future development of a patient's skin condition. The XR imagery may include “live” or “real-time” augmented reality (AR) imagery of the patient's body overlaid with a virtual three-dimensional (3-D) model of the predicted skin condition, or in other examples, a virtual 3-D model of the predicted skin condition overlaid on the patient's actual body as viewed through a transparent display screen.

As one non-limiting example, the techniques of this disclosure include a computing system configured to capture sensor data (including 2-D image data) indicative of a patient's skin condition, feed the collected data through a deep-learning model configured to estimate the skin-condition type, predict a unique future development of the skin condition, and generate and output XR imagery visualizing the predicted future development of the skin condition. In this way, the techniques described herein may provide one or more technical advantages that provide at least one practical application. For example, the techniques described in this disclosure may be configured to provide more accurate and/or comprehensive visual information to a specialist (e.g., a dermatologist).

In some additional aspects, the techniques of this disclosure describe improved techniques for generating the XR elements as compared to more-typical techniques. As one example, the techniques of this disclosure include generating and rendering XR elements (e.g., three-dimensional virtual models) based on 3-D sensor data as input, thereby enabling more-accurate virtual imagery (e.g., 3-D models) constructed over a framework of curved surfaces, as compared to more-common planar surfaces.

In one example, the techniques described herein include a method performed by a computing system, the method comprising: estimating, based on sensor data, a skin-condition type for a skin condition on an affected area of a body of a patient; determining, based on the sensor data and the estimated skin-condition type, modeling data indicative of a typical development of the skin-condition type; generating, based on the sensor data and the modeling data, a 3-dimensional (3-D) model indicative of a predicted future development of the skin condition over time; generating extended reality (XR) imagery of the affected area of the body of the patient overlaid with the 3-D model; and outputting the XR imagery.

In another example, the techniques described herein include a computing system comprising processing circuitry configured to: estimate, based on sensor data, a skin-condition type for a skin condition on an affected area of a body of a patient; determine, based on the sensor data and the estimated skin-condition type, modeling data indicative of a typical development of the skin-condition type; generate, based on the sensor data and the modeling data, a 3-dimensional (3-D) model indicative of a predicted future development of the skin condition over time; generate extended reality (XR) imagery of the affected area of the body of the patient overlaid with the 3-D model; and output the XR imagery.

In another example, the techniques described herein include a non-transitory computer-readable medium comprising instructions for causing one or more programmable processors to: estimate, based on sensor data, a skin-condition type for a skin condition on an affected area of a body of a patient; determine, based on the sensor data and the estimated skin-condition type, modeling data indicative of a typical development of the skin-condition type; generate, based on the sensor data and the modeling data, a 3-dimensional (3-D) model indicative of a predicted future development of the skin condition over time; generate extended reality (XR) imagery of the affected area of the body of the patient overlaid with the 3-D model; and output the XR imagery.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram depicting an example skin-condition-prediction system, in accordance with the techniques of this disclosure.

FIG. 2A is a block diagram depicting an example computing system configured to predict a dermatological condition, in accordance with one or more aspects of the techniques disclosed.

FIG. 2B is a block diagram depicting an example hardware architecture of the computing system of FIG. 2A.

FIG. 2C is a block diagram depicting example software modules of the computing system of FIG. 2A.

FIGS. 3A-3D are conceptual diagrams illustrating techniques for predicting and visualizing a development of a skin condition, in accordance with one or more aspects of the techniques disclosed.

FIG. 4 is a conceptual diagram depicting an example of the skin-condition-prediction system of FIG. 1.

FIG. 5 is a flowchart illustrating an example skin-condition-prediction process, in accordance with one or more aspects of the techniques disclosed.

FIG. 6 is a flowchart illustrating another example skin-condition-prediction process, in accordance with one or more aspects of the techniques disclosed.

### DETAILED DESCRIPTION

A dermatological patient may suffer from a skin condition, such as a rash, burn, abrasion, outbreak, blemish, bruise, infection, tumor, lesions, necrosis, boils, blisters, discoloration, or the like. In the absence of treatment, or similarly, in the presence of incorrect or ineffective treatment (as a result of, for example, incorrect diagnosis), the condition may grow, spread, or otherwise change over time. Advances in artificial intelligence (AI), deep learning (DL), and machine-learning systems and techniques may enable systems to be trained to estimate (e.g., identify, to a certain probability) the skin-condition type or category based on 2-D imagery of the condition. For example, with the development of high-performance graphics processing units (GPUs) and specialized hardware for AI, the machine-learning field may be developed to implement various pattern-recognition architectures in neural networks (NNs) in order to classify (e.g., categorize, label, or identify) a condition based on a two-dimensional (2-D) image of an affected skin area.

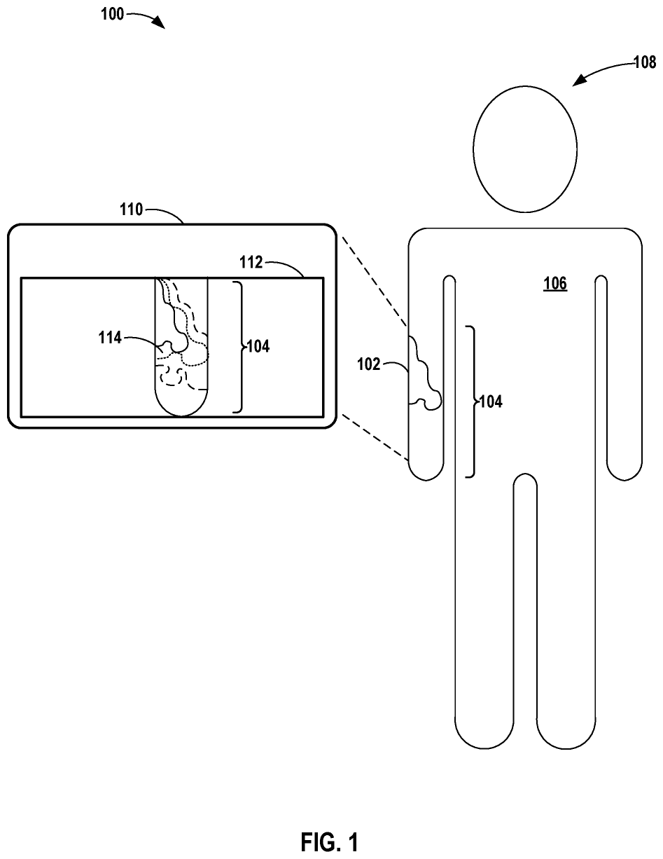

According to techniques of this disclosure, a computing system (e.g., one or more computing devices) may be configured to not only estimate a skin-condition type with greater accuracy and precision than existing techniques (e.g., due to, inter alia, a more comprehensive set of sensor-data input), but also to predict and visualize a future development of the skin condition over time. For example, FIG. 1 depicts a conceptual diagram of a skin-condition-prediction system 100 configured to predict and visualize a future development of a skin condition 102 on an affected area or region 104 of a body 106 of a patient 108, in accordance with techniques of this disclosure.

In general, system 100 represents or includes a computing system 110 configured to estimate (e.g., determine or identify, to a certain probability), based on sensor data, a skin-condition type, label, or category corresponding to skin condition 102. Computing system 110 may further determine (e.g., retrieve, receive, generate, etc.), based on the sensor data and the estimated type of skin condition 102, modeling data indicative of a typical development of the estimated type of skin condition 102. Computing system 110 may then generate, based on the sensor data and the modeling data, a three-dimensional (3-D) model indicative of a predicted future development of skin condition 102 over time; generate extended-reality (“XR”) imagery 112 of the patient's affected skin area 104 overlaid with the 3-D model; and output the XR imagery 112 for display.

As used herein, the term “extended reality” encompasses a spectrum of user experiences that includes virtual reality (“VR”), mixed reality (“MR”), augmented reality (“AR”), and other user experiences that involve the presentation of at least some perceptible elements as existing in the user's environment that are not present in the user's real-world environment, as explained further below. Thus, the term “extended reality” may be considered a genus for MR, AR, and VR.

“Mixed reality” (MR) refers to the presentation of virtual objects such that a user sees images that include both real, physical objects and virtual objects. Virtual objects may include text, 2-D surfaces, 3-D models, or other user-perceptible elements that are not actually present in the physical, real-world environment in which they are presented as coexisting. In addition, virtual objects described in various examples of this disclosure may include graphics, images, animations or videos, e.g., presented as 3-D virtual objects or 2-D virtual objects. Virtual objects may also be referred to as “virtual elements.” Such elements may or may not be analogs of real-world objects.

In some examples of mixed reality, a camera may capture images of the real world and modify the images to present virtual objects in the context of the real world. In such examples, the modified images may be displayed on a screen, which may be head-mounted, handheld, or otherwise viewable by a user. This type of MR is increasingly common on smartphones, such as where a user can point a smartphone's camera at a sign written in a foreign language and see in the smartphone's screen a translation in the user's own language of the sign superimposed on the sign along with the rest of the scene captured by the camera. In other MR examples, in MR, see-through (e.g., transparent) holographic lenses, which may be referred to as waveguides, may permit the user to view real-world objects, i.e., actual objects in a real-world environment, such as real anatomy, through the holographic lenses and also concurrently view virtual objects.

The Microsoft HOLOLENS™ headset, available from Microsoft Corporation of Redmond, Wash., is an example of a MR device that includes see-through holographic lenses that permit a user to view real-world objects through the lens and concurrently view projected 3D holographic objects. The Microsoft HOLOLENS™ headset, or similar waveguide-based visualization devices, are examples of an MR visualization device that may be used in accordance with some examples of this disclosure. Some holographic lenses may present holographic objects with some degree of transparency through see-through holographic lenses so that the user views real-world objects and virtual, holographic objects. In some examples, some holographic lenses may, at times, completely prevent the user from viewing real-world objects and instead may allow the user to view entirely virtual environments. The term mixed reality may also encompass scenarios where one or more users are able to perceive one or more virtual objects generated by holographic projection. In other words, “mixed reality” may encompass the case where a holographic projector generates holograms of elements that appear to a user to be present in the user's actual physical environment.

In some examples of mixed reality, the positions of some or all presented virtual objects are related to positions of physical objects in the real world. For example, a virtual object may be tethered or “anchored” to a table in the real world, such that the user can see the virtual object when the user looks in the direction of the table but does not see the virtual object when the table is not in the user's field of view. In some examples of mixed reality, the positions of some or all presented virtual objects are unrelated to positions of physical objects in the real world. For instance, a virtual item may always appear in the top-right area of the user's field of vision, regardless of where the user is looking. XR imagery or visualizations may be presented in any of the techniques for presenting MR, such as a smartphone touchscreen.

Augmented reality (“AR”) is similar to MR in the presentation of both real-world and virtual elements, but AR generally refers to presentations that are mostly real, with a few virtual additions to “augment” the real-world presentation. For purposes of this disclosure, MR is considered to include AR. For example, in AR, parts of the user's physical environment that are in shadow can be selectively brightened without brightening other areas of the user's physical environment. This example is also an instance of MR in that the selectively brightened areas may be considered virtual objects superimposed on the parts of the user's physical environment that are in shadow.

Furthermore, the term “virtual reality” (VR) refers to an immersive artificial environment that a user experiences through sensory stimuli (such as sights and sounds) provided by a computer. Thus, in VR, the user may not see any physical objects as they exist in the real world. Video games set in imaginary worlds are a common example of VR. The term “VR” also encompasses scenarios where the user is presented with a fully artificial environment, in which the locations of some virtual objects are based on the locations of corresponding physical objects relative to the user. Walk-through VR attractions are examples of this type of VR. XR imagery or visualizations may be presented using techniques for presenting VR, such as VR goggles.

In accordance with techniques of this disclosure, computing system 110 is configured to generate and output XR imagery 112 of a predicted future development of skin condition 102 of patient 108. In some examples, XR imagery 112 may include “live” or “real-time” composite 2-D imagery of the affected region 104 of the patient's body 106, overlaid with a projection of a virtual 3-D model 114 of the predicted skin-condition development. In other examples, XR imagery 112 may include the projection of the virtual 3-D model 114 displayed relative to the affected area 104 of the patient's actual body 106, as viewed through a transparent display screen.

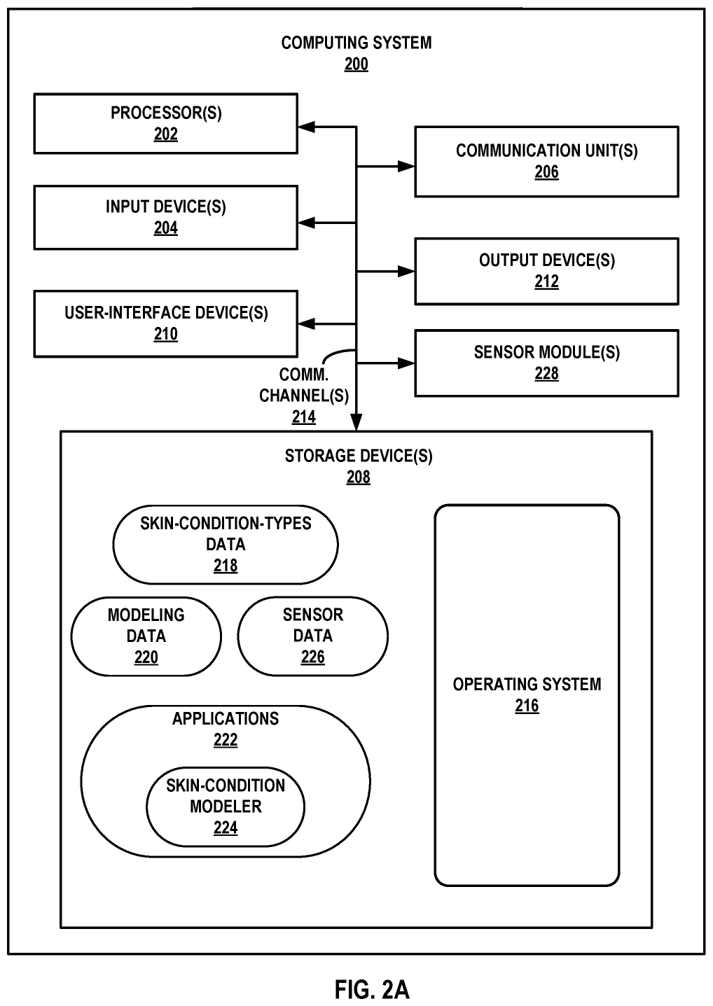

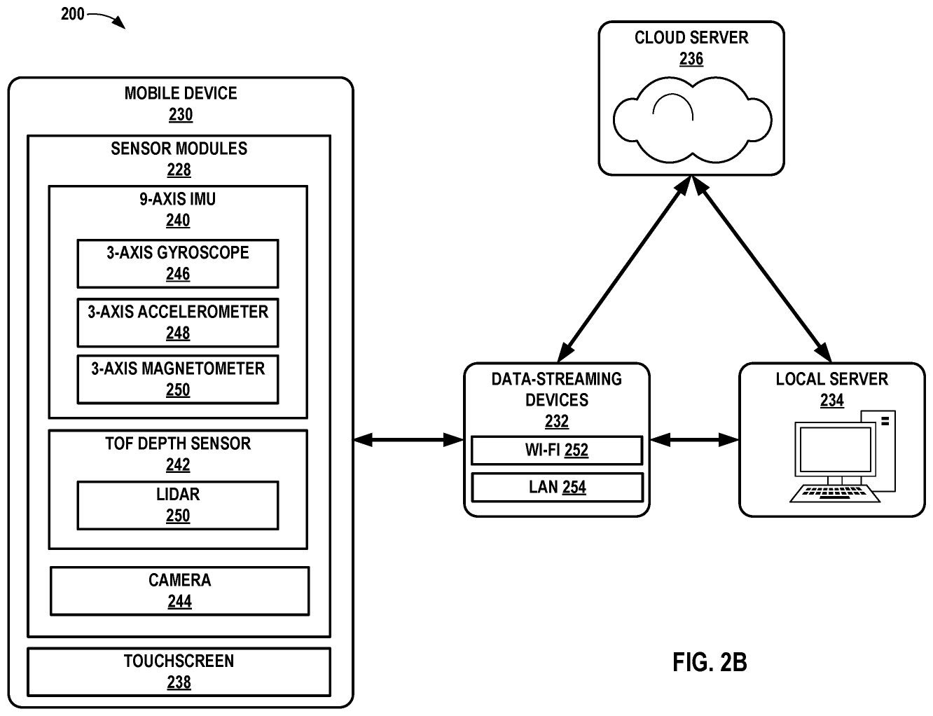

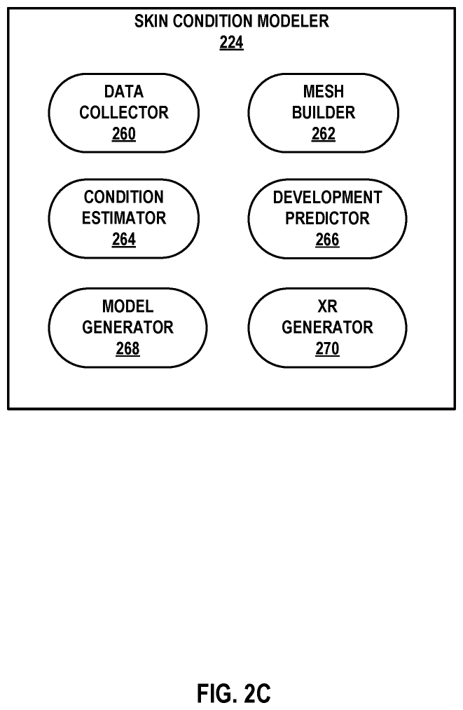

FIG. 2A is a block diagram of an example computing system 200 that operates in accordance with one or more techniques of the present disclosure. FIG. 2A may illustrate a particular example of computing system 110 of FIG. 1. In other words, computing system 200 includes one or more computing devices, each computing device including one or more processors 202, any or all of which are configured to predict and visualize a future development of skin condition 102 of patient 108 (FIG. 1).

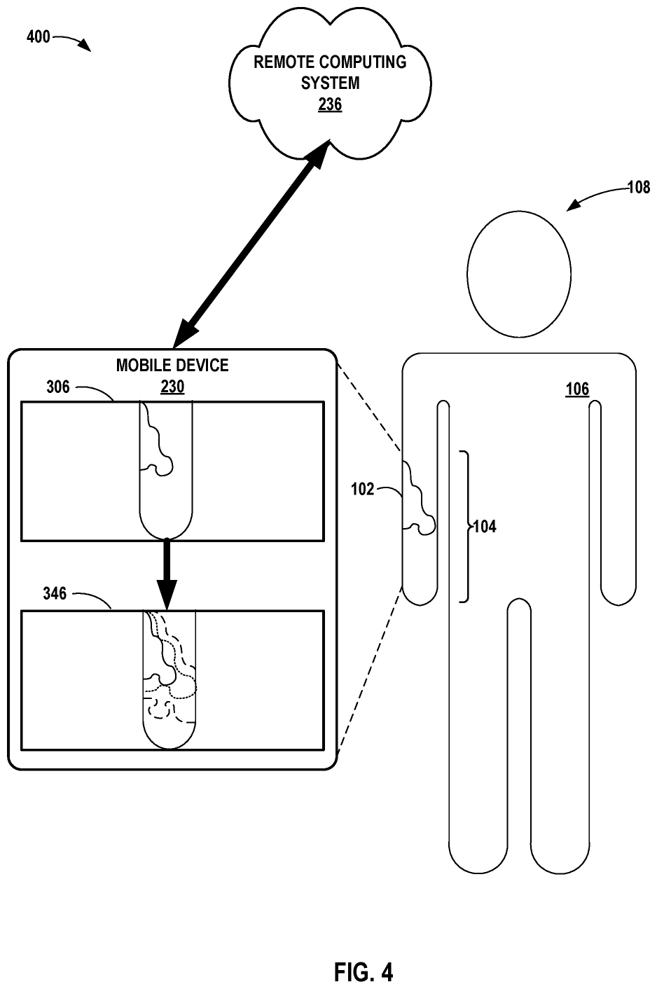

As detailed further below with respect to the example hardware architectures depicted in FIG. 2B and FIG. 4, computing system 200 of FIG. 2A may include one or more of a workstation, server, mainframe computer, notebook or laptop computer, desktop computer, tablet, smartphone, XR display device, datastore, distributed network, and/or other programmable data-processing apparatuses of any kind. In some examples, a computing system may be or may include any component or system that includes one or more processors or other suitable computing environment for executing software instructions configured to perform the techniques described herein, and, for example, need not necessarily include one or more elements shown in FIG. 2A. As one illustrative example, communication units 206, and in some examples, other components such as storage device(s) 208, may not necessarily be included within computing system 200, in examples in which the techniques of this disclosure may be performed without these components.

As shown in the specific example of FIG. 2A, computing system 200 includes one or more processors 202, one or more input devices 204, one or more communication units 206, one or more output devices 212, one or more storage devices 208, one or more user interface (UI) devices 210, and in some examples, but not all examples, one or more sensor modules 228 (also referred to herein as “sensors 228”). Computing system 200, in one example, further includes one or more applications 222 and operating system 216 (e.g., stored within a computer readable medium, such as storage device(s) 208) that are executable by processors 202 of computing system 200.

Each of components 202, 204, 206, 208, 210, 212, and 228 is coupled (physically, communicatively, and/or operatively) for inter-component communications. In some examples, communication channels 214 may include a system bus, a network connection, an inter-process communication data structure, or any other method for communicating data. As one example, components 202, 204, 206, 208, 210, 212, and 228 may be coupled by one or more communication channels 214. In some examples, two or more of these components may be distributed across multiple (discrete) computing devices. In some such examples, communication channels 214 may include wired or wireless data connections between the various computing devices.

Processors 202, in one example, are configured to implement functionality and/or process instructions for execution within computing system 200. For example, processors 202 may be capable of processing instructions stored in storage device 208. Examples of processors 202 may include one or more of a microprocessor, a controller, a digital signal processor (DSP), an application-specific integrated circuit (ASIC), a field-programmable gate array (FPGA), or equivalent discrete or integrated logic circuitry.

One or more storage devices 208 (also referred to herein as “memory 208”) may be configured to store information within computing system 200 during operation. Storage device(s) 208, in some examples, are described as computer-readable storage media. In some examples, storage device 208 is a temporary memory, meaning that a primary purpose of storage device 208 is not long-term storage. Storage device 208, in some examples, is described as a volatile memory, meaning that storage device 208 does not maintain stored contents when the computer is turned off. Examples of volatile memories include random access memories (RAM), dynamic random access memories (DRAM), static random access memories (SRAM), and other forms of volatile memories known in the art.

In some examples, storage device 208 is used to store program instructions for execution by processors 202. Storage device 208, in one example, is used by software or applications running on computing system 200 to temporarily store information during program execution. For example, as shown in FIG. 2A, storage device 208 is configured to store operating system 216, skin-condition-types data 218, modeling data 220, sensor data 226, and various programs or applications 222, including a skin-condition modeler 224, as detailed further below with respect to FIG. 2C.

Storage devices 208, in some examples, also include one or more computer-readable storage media. Storage devices 208 may be configured to store larger amounts of information than volatile memory. Storage devices 208 may further be configured for long-term storage of information. In some examples, storage devices 208 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing system 200, in some examples, also includes one or more communication units 206. Computing system 200, in one example, utilizes communication units 206 to communicate with external devices via one or more networks, such as one or more wired/wireless/mobile networks. Communication unit(s) 206 may include a network interface card, such as an Ethernet card, an optical transceiver, a radio frequency transceiver, or any other type of device that can send and receive information. Other examples of such network interfaces may include 3G, 4G, 5G and Wi-Fi radios. In some examples, computing system 200 uses communication unit 206 to communicate with an external device.

Computing system 200, in one example, also includes one or more user-interface (“UI”) devices 210. UI devices 210, in some examples, are configured to receive input from a user through tactile, audio, or video feedback. Examples of UI device(s) 210 include a presence-sensitive display, a mouse, a keyboard, a voice-responsive system, a video camera, a microphone, or any other type of device for detecting a command from a user. In some examples, a presence-sensitive display includes a touch-sensitive screen or “touchscreen.”

One or more output devices 212 may also be included in computing system 200. Output device 212, in some examples, is configured to provide output to a user using tactile, audio, or video stimuli. Output device 212, in one example, includes a presence-sensitive display, a sound card, a video graphics adapter card, or any other type of device for converting a signal into an appropriate form understandable to humans or machines. Additional examples of output device 212 include a speaker, a cathode ray tube (CRT) monitor, a liquid crystal display (LCD), or any other type of device that can generate intelligible output to a user.

Computing system 200 may include operating system 216. Operating system 216, in some examples, controls the operation of components of computing system 200. For example, operating system 216, in one example, facilitates the communication of one or more applications 222 with processors 202, communication unit 206, storage device 208, input device 204, user interface device 210, and output device 212.

Application 222 may also include program instructions and/or data that are executable by computing system 200. As detailed below with respect to FIG. 2C, skin-condition modeler 224 is one example of an application 222 of computing system 200. For instance, skin-condition modeler 224 may include instructions for causing computing system 200 to perform techniques described in the present disclosure, for example, to predict and visualize a future development of skin condition 102 of patient 108 (FIG. 1).

FIG. 2B is a block diagram depicting individual computing devices of an example hardware architecture of computing system 200 of FIG. 2A. As shown in FIG. 2B, computing system 200 includes a mobile device 230, one or more data-streaming devices 232, a local server 234, and a cloud server 236. The example hardware architecture depicted in FIG. 2B is intended for illustrative purposes only, and is not intended to be limiting. Other architectures of system 200 of FIG. 2A having more, fewer, or different computing devices than those depicted in FIG. 2B may likewise be configured to perform techniques of this disclosure.

For instance, other examples of hardware architectures of computing system 200 may include a physically distinct XR-display device, such as an MR or VR headset. In other examples, such as the example depicted in FIG. 2B, the functionality of an XR-display device may be performed by mobile device 230, as detailed further below. As another example, the functionality of local server 234 may be performed by mobile device 230. Accordingly, any of the techniques described herein as being performed by either mobile device 230 or local server 234 may, in fact, be performed by the other device or by both devices.

Mobile device 238 may include virtually any mobile (e.g., lightweight and portable) computing device that is local to a user. For example, mobile device 238 may include a smartphone, tablet, or the like, that includes sensor modules 228 (or “sensors 228”) and a display screen 238. As detailed further below, sensors 228 are configured to capture sensor data 226 indicative or descriptive of skin condition 102 of patient 108 of FIG. 1. Sensor modules 228 of mobile device 238 may include, as non-limiting examples, an inertial measurement unit (IMU) 240, a camera 244, and in some examples, but not all examples, depth sensor 242.

In the specific example depicted in FIG. 2B, IMU 240 includes a 9-axis IMU including: a 3-axis gyroscope 246 configured to generate angular rate data indicating a change in position of mobile device 230; a 3-axis accelerometer 248 configured to capture data indicative of an acceleration of mobile device 230 due to outside forces; and a 3-axis magnetometer 250 configured to determine the orientation of mobile device 230 relative to Earth's magnetic field.

In some examples, depth sensor 242 may include a time-of-flight (TOF)-based depth sensor configured to measure a distance to an object by reflecting a signal off of the object and measuring the duration between transmission of the initial signal and receipt of the reflected signal. In the specific example depicted in FIG. 2B, depth sensor 242 includes light-detection-and-ranging (LIDAR) 250, configured to generate infrared (IR) depth data configured to indicate the distance between the body 106 of patient 108 (FIG. 1) and mobile device 230.

Camera 244 is configured to capture standard red-green-blue (RGB) image data. As one non-limiting example, camera 244 may include an integrated 4-Megapixel camera configured to capture images at about 30 to 60 frames per second (FPS).

Display screen 238, which is an example of UI device 210 of FIG. 2A, is configured to output XR content 112 (FIG. 1) for display to a user of mobile device 230. Display screen 238 may include a touchscreen, transparent visor, or other similar surface configured to display graphical content.

Data-streaming devices 232 may be examples of communication channels 214 of FIG. 2A. As shown in FIG. 2B, data-streaming devices 232 may include Wi-Fi 252, local-area-network (LAN) connections 254, and/or other hardware fabricated according to appropriate data-communication protocols to transfer data between the various computing devices of computing system 200.

In some examples, but not all examples, local server 234 may include any suitable computing device (e.g., having processing circuitry and memory) that is physically or geographically local to a user of mobile device 230. As one non-limiting example, local server 234 may include a CUDA-enabled graphics-processing unit (GPU); an Intel i7+ processor; and installed software including Nvidia's CUDA and CUDNN (10.1 or later), Python, C#, and CUDA C++. In other examples, as referenced above, local server 234 may be integrated within mobile device 230, such that mobile device 230 may perform the functionality ascribed to both devices. In some such examples, local server 230 may be conceptualized as a “module” (e.g., one or more applications) running on mobile device 230 and configured to provide a “service” according to techniques of this disclosure.

Cloud server 236 includes any computing device(s) (e.g., datastores, server rooms, etc.) that are not geographically local to a user of mobile device 230 and local server 234. For instance, in examples in which mobile device 230 includes an “activated” smartphone, cloud server 236 may include remote computing servers managed by the telecommunications network configured to provide cellular data to mobile device 230, and/or computing servers managed by developers of applications 222 (e.g., skin-condition modeler 224) running on mobile device 230.

FIG. 2C is a block diagram illustrating example software modules of computing system 200 of FIG. 2A, and more specifically, illustrating example sub-modules of skin-condition modeler 224. For illustrative purposes, and for ease of understanding, the functionality of the software modules of FIG. 2C are described with reference to the example hardware architecture depicted in FIG. 2B. As shown in FIG. 2C, skin-condition modeler 224 includes data collector 260, mesh builder 262, condition estimator 264, development predictor 266, model generator 268, and XR generator 270. In other examples, skin-condition modeler 224 may include more, fewer, or different software components configured to perform techniques in accordance with this disclosure.

In some examples, but not all examples, skin-condition modeler 224 is configured to passively receive a comprehensive set of sensor data 226 describing or otherwise indicative of various aspects of skin condition 102. In other examples, skin-condition modeler 224 includes data collector 260, a module configured to actively retrieve, aggregate, and/or correlate sensor data 226. For example, data collector 260 may be in data communication with sensors 228 that are physically integrated within mobile device 230 (or other computing device of computing system 200) and/or other physically distinct sensor modules that are communicatively coupled to computing system 200. In some such examples, data collector 260 is configured to control sensor modules 228, e.g., to command the sensors 228 to generate and output sensor data 226.

As described above with respect to FIG. 2B, sensor data 226 may include a variety of different types of sensor data, such as, but not limited to, motion and orientation data from IMU 240, relative depth data from depth sensor 242, and 2-D image data from camera 244. In some examples, the 2-D image data includes a plurality of overlapping 2-D images of the affected area 104 of the body 106 of patient 108 that collectively define 3-D, arcuate-shaped imagery.

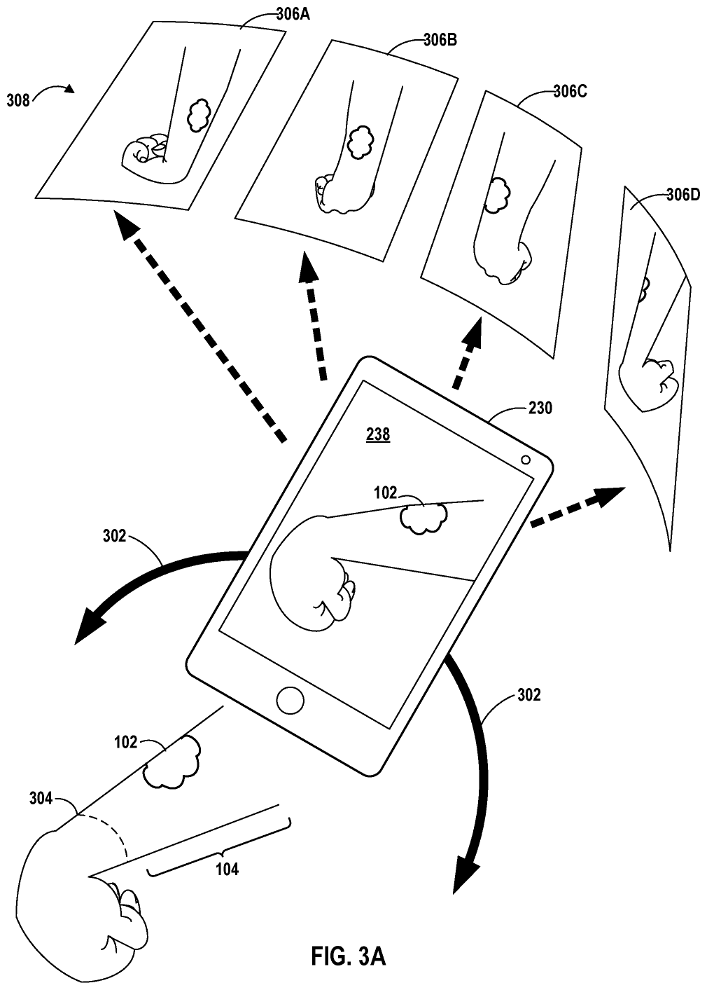

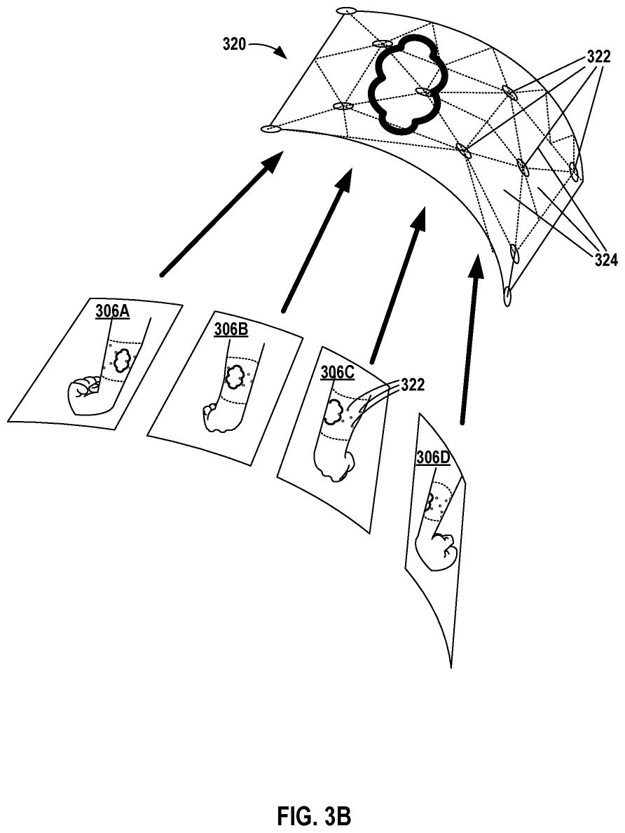

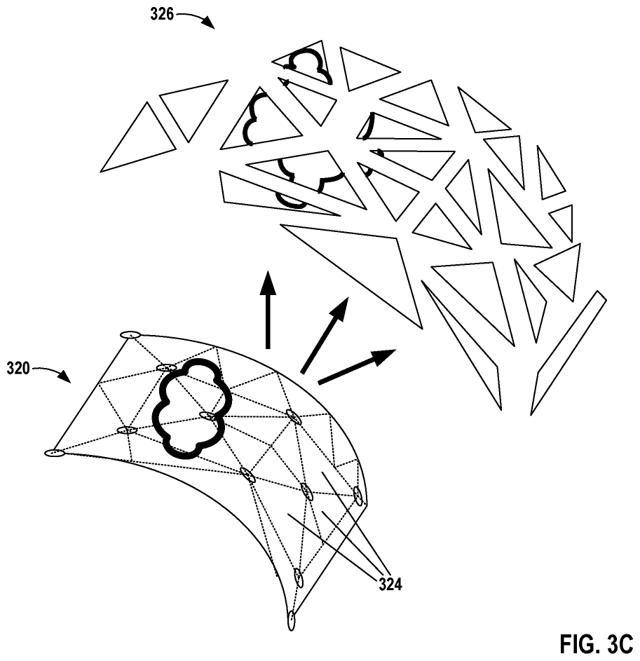

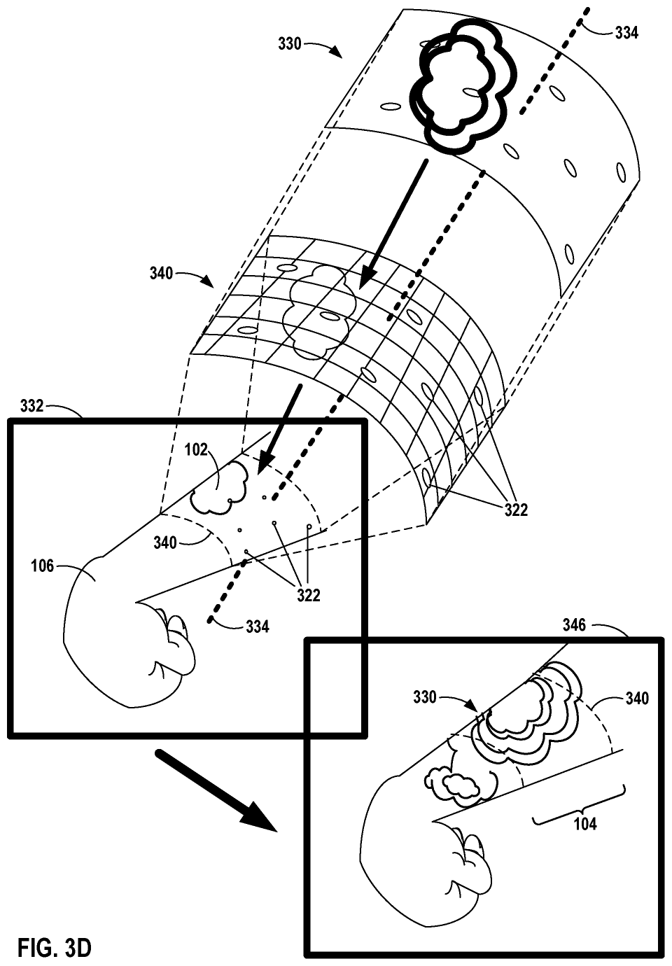

For instance, as illustrated in FIG. 3A, image data of sensor data 226 may include a plurality of overlapping 2-D images 306A-306D (collectively “2-D images 306”) captured by camera 244 of mobile device 230, while mobile device 230 moves along an arcuate-shaped path of motion, such that, when aligned according to their respective location and orientation of capture, the 2-D images 306 collectively define a conceptual curved surface 308. As one non-limiting example, 2-D images 306 may be collected by a user, such as a clinician of patient 108 (e.g., a dermatologist), the patient 108 themselves, or another user of mobile device 230, by moving mobile device 230 in an arcuate (e.g., curved) motion, as indicated by arrows 302 in FIG. 3A. For instance, the user may move mobile device 230 along a curved path 302 that generally correlates to a curvature 304 of the affected area 104 of the patient's body 106, in order to capture imagery of skin condition 102 from multiple angles along the curvature 304 of the patient's body 106. For example, the user may revolve mobile device 230 along a 180-degree arc centered on the affected area 104 of the patient's body 106, while keeping the lens of camera 244 aimed at (e.g., directed toward) the affected area 104.

In alternate examples in which camera 244 is not integrated within mobile device 230, data collector 260 may be configured to control a specialized image-capture device that is specifically designed to capture 2-D images 306 along an arcuate path of motion. One illustrative example of such an image-capture device is an orthodontist's dental x-ray machine, which revolves an x-ray emitter and an x-ray detector around the curvature of a patient's head while capturing x-ray imagery at a plurality of different positions along the path of motion.

While camera 244 captures 2-D images 306 along curved path 302, one or more additional sensors 228 (e.g., IMU 240 and/or depth sensor 242) simultaneously collect other types of sensor data 226 that may be correlated to the 2-D images 306. For example, data collector 260 may use IMU data from IMU 240 to determine, for each 2-D image 306, a viewing angle (e.g., orientation) of camera 244 relative to, for example, Earth's gravity and Earth's magnetic field, and by extension, relative to a prior image and/or a subsequent image of 2-D images 306. Similarly, data collector 260 may use depth data from depth sensor 242 to determine, for each 2-D image 306, a relative distance between the affected area 104 of patient 108 (as depicted within each 2-D image) and camera 244, and by extension, a relative location of mobile device 230 when each 2-D image 306 was captured.

In examples in which the individual types of sensor data are not already (e.g., automatically) associated in this way upon capture, data collector 260 may be configured to correlate or aggregate the various types of sensor data to produce correlated datasets, wherein each dataset includes sensor data 226 from different types of sensors 228, but that was captured at approximately the same instance in time (e.g., within a threshold range or “window” of time). For instance, data collector 260 may embedded timestamp data in order to produce the correlated datasets. Data collector 260 may then transfer a copy of the correlated sensor data 226 to mesh builder 262.

In general, as illustrated in FIG. 3B, mesh builder 262 is configured to use sensor data 226 to generate, based on sensor data 226 (e.g., based at least in part on 2-D images 306), a virtual, 3-D curved polygon mask 320 that graphically represents the patient's affected skin area 104. For example, mesh builder 262 may be configured to analyze the 2-D images 306 in order to identify a plurality of feature points 322 within the 2-D images 306. Feature points 322 may include virtually any identifiable object or landmark appearing in at least two overlapping images of 2-D images 306. In some examples, feature points 322 may include, as non-limiting examples, a freckle, an edge or outline of the patient's body 106, an edge or outline of skin condition 102, or a sub-component of skin condition 102, such as an individual bump or spot.

After identifying feature points 322, mesh builder 262 may attempt to match corresponding (e.g., identical) feature points across two or more overlapping images of the 2-D images 306. In some examples, but not all examples, mesh builder 262 may then use the relative (2-D) positions of feature points 322 within the respective 2-D images to orient (e.g., align) the 2-D images 306 relative to one another, and by extension, the graphical image content (e.g., the patient's affected skin area 104) contained within the 2-D images 306.

Mesh builder 262 may use the correlated sensor data 226 (e.g., depth-sensor data and/or IMU data), to determine a relative 3-D position of each feature point relative to the other feature points 322. Mesh builder 322 may then draw (e.g., define) a virtual “edge” between each pair of adjacent or proximal feature points 322, thereby defining a plurality of 2-D polygons 324 or “tiles” that collectively define 3-D polygon mask 320 having a curvature that accurately represents (e.g., highly conforms to) the curved geometry 304 of the affected skin area 104 of the patient's body.

In this way, mesh builder 262 reduces an amount of distortion that would otherwise appear in any single 2-D image 306 depicting skin condition 102. For example, analogous to how projecting the surface of a globe onto a 2-D map of planet Earth results in increasingly distorted continents at latitudes farther from the Equator, capturing a 2-D image 306 of a curved area 104 of a patient's body 106 inherently distorts and/or obscures any portion of the curved area that is not directly tangent to an optical axis of the camera 244. Accordingly, any skin-condition-estimation technique based directly on captured 2-D images naturally introduces a significant amount of error when attempting to recognize a distorted pattern or texture of the skin condition. However, in the techniques described herein, mesh builder 262 essentially assembles 3-D polygon mesh 320 by identifying and extracting relatively un-distorted sections within 2-D images 306 (e.g., portions of 2-D images 306 that were oriented generally perpendicular to the optical axis of camera 244 at the time of capture), and assembling the extracted un-distorted image sections into a relatively high-resolution virtual 3-D model of affected skin area 104. Mesh builder 262 may then transfer a copy of 3-D polygon mask 320 to condition estimator 264.

In general, condition estimator 264 is configured to determine, based at least in part on 3-D polygon mask 320 derived from sensor data 226, a skin-condition “type” (e.g., category or label) that matches, represents, defines, or otherwise applies to the patient's skin condition 102, to within a certain (e.g., above-threshold) probability. For example, as used herein, a skin-condition “type” may refer to, as non-limiting examples: (1) a broad or general category of skin conditions (e.g., “rash” or “blemish”); (2) a specific medical name for a skin condition or a group of related skin conditions (e.g., “folliculitis”); (3) a determinable cause of a skin condition (e.g., “mosquito bite” or “scabies”); or (4) any other similar label corresponding to a set of objective descriptive parameters of (e.g., criteria for) a known skin condition, such that a determined applicable label provides useful information about the patient's skin condition 102.

In some examples, condition estimator 264 may be configured to generate, based on 3-D polygon mask 320, “revised” 2-D imagery that more accurately depicts the patient's skin condition 102 (e.g., with significantly reduced image distortion, as described above) than any individual 2-D image of sensor data 226, and then estimate an applicable skin-condition type based on the revised 2-D imagery.

For instance, as illustrated conceptually in FIG. 3C, condition estimator 264 may decompose the surface (e.g., the color and texture data overlying the virtual polygon structure) of 3-D polygon mask 320 into revised 2-D imagery 326. For example, as described above, the “faces” of polygons 324 are extracted sections of 2-D images 306 that most-accurately depict (e.g., with the least distortion) the texture, color, etc., of the patient's affected skin area 104. Because the curvature 304 of the patient's body, and by extension, the corresponding curvature of 3-D polygon mask 320, is not particularly relevant to identifying the skin-condition type, condition estimator 264 is configured to analyze the un-distorted image data of the individual 2-D polygons 324, irrespective of the relative orientations between the polygons. Accordingly, in some examples, condition estimator 264 may “flatten” the polygons 324 onto a single planar surface, such as into a single common 2-D image or imagery 326, in order to perform texture-based and pattern-based analysis of the 2-D polygons 324. As referenced above, in this way (e.g., via a 2-D-to-3-D-to-2-D image-conversion technique), condition estimator 264 produces a substantially high-resolution (e.g., minimal-distortion) representation of skin condition 102 on which to base an estimation of a matching skin-condition type.

In some examples, but not all examples, when flattening 3-D polygon mask 320 into 2-D imagery 326, condition estimator 264 may be configured to intentionally re-introduce a minor amount of distortion of polygons 324. For example, in order to extrapolate (e.g., approximate) a shape (e.g., perimeter or outline) of skin condition 102 for purposes of estimating the skin-condition type (such as for smaller, local sub-sections of the affected area 104), condition estimator 264 may “fill-in” the gaps between individual polygons 324, such as by replicating the texture or pattern of the adjacent polygons into the gaps. In other examples, condition estimator 264 may analyze the texture, pattern, and/or color each polygon individually of the other polygons 324, thereby abrogating the need to extrapolate pixels between consecutive polygons.

In some examples, but not all examples, prior to determining a matching skin-condition type, condition estimator 264 may be configured to automatically identify (e.g., locate) the affected area 104 of the patient's body 106, either within the original 2-D images 306 from camera 244 or on the surface of the 3-D polygon mask 320. For example, in response to user input, condition estimator 264 may automatically perform texture-and-color analysis on 2-D images 306 (e.g., “image data”) in order to locate the affected area 104 within the 2-D images 306 or within 3-D polygon mask 320, as appropriate. For instance, condition estimator 264 may apply one or more pattern-recognition algorithms to the image data in order to identify and return an area or areas of the image data that have characteristics typical of skin conditions, including, as non-limiting examples, reddish or darkish coloration, a raised texture indicating hives or bumps, or any other abrupt transition in continuity of color or pattern on the patient's body, indicating a rash or lesion.

In other examples, such as examples in which sensors 228 include an infrared-based depth sensor 250, condition estimator 234 may identify (e.g., locate) the affected area based on infrared data. For example, the patient's body 108 may appear “warmer” than the surrounding environment within the infrared data. Accordingly, condition estimator 264 may use the infrared data to “narrow down” the set of potential skin-condition locations to areas including the patient's body 108, and then use other image-recognition techniques to particularly locate the affected skin area 104.

In other examples, condition estimator 264 may identify the affected area 104 of the patient's body 106 based on user input. For example, skin-condition modeler 224 may prompt the user to indicate, such as by using a finger or by drawing a bounding box on display screen 236 of mobile device 230, the location of affected area 104 within one of 2-D images 306 or on 3-D polygon mask 320 displayed on display screen 238.

Condition estimator 264 may determine a matching skin-condition type, such as by comparing 2-D imagery 326 (and/or sensor data 226) to a set of skin-condition-types data 218 (e.g., retrieved from storage device(s) 208 of FIG. 2A). In some examples, skin-condition-types data 218 may include, for each of a plurality of different types of known skin conditions, data indicative of a typical physical appearance or other common physical attributes of the respective skin condition. In some examples, skin-condition-types data 218 may include, for each type of skin condition, an objectively defined range of values for each of a plurality of skin-condition parameters. For instance, example skin-condition parameters may include a relative coloring, a pattern (e.g., color pattern), a texture (e.g., physical pattern), a size, a shape, or any other objectively identifiable and measurable quality of the skin condition. As one illustrative example, skin-condition-types data 218 may describe a particular type of skin condition that includes a bumpy-texture parameter, wherein the dataset for that skin-condition type includes a range of values defining a typical density of bumps per unit surface area of skin, or a range of values defining typical diameters of each bump.

In some examples, the “typical” value or values for a skin-condition parameter includes a simple numerical range (e.g., from 6-10 bumps per square inch). In some such examples, by comparing 2-D imagery 326 to skin-condition-types data 218, condition estimator 264 may return a plurality of different “candidate” skin-condition types, wherein the patient's skin condition 102 satisfies the criteria (e.g., falls within the ranges of parameter values) for every candidate skin-condition type.

In other examples, the “typical” value or values for a skin-condition parameter includes a Gaussian or “normal” probability distribution indicating relative probabilities of different values, such as based on a number of standard deviations from a most-probable value. In some such examples, condition estimator 264 may be configured to select or identify a single best-matched skin-condition type, wherein the patient's skin condition 102 most-approximates the most-probable value across the various indicated parameters for the best-matched skin-condition type.

In some examples, skin-condition-types data 218 may include one or more parameters based on other sensor data 226, such as infrared data from depth-sensor 242. As one illustrative example, infrared data may indicate a particularly “warm” region of the patient's body 108, which, as indicated within skin-condition-types data 218, may be indicative of a skin-condition type such as “recent burn” or other typically exothermic skin condition.

In the above-described examples, condition estimator 264 identifies one or more matching types of skin conditions based on objective, articulable criteria that may be readily available to a user of computing system 200, if desired. In other words, computing system 200 may be configured to output a report articulating the objective basis for the determined skin-condition type.

In other examples, condition estimator 264 may include one or more artificial-intelligence (AI), deep-learning, or machine-learning models or algorithms configured to determine or estimate a skin-condition type that matches the patient's skin condition 102 based on 2-D imagery 326. In general, a computing system uses a machine-learning algorithm to build a model based on a set of training data such that the model “learns” how to make predictions, inferences, or decisions to perform a specific task without being explicitly programmed to perform the specific task. Once trained, the computing system applies or executes the trained model to perform the specific task based on new data. Examples of machine-learning algorithms and/or computer frameworks for machine-learning algorithms used to build the models include a linear-regression algorithm, a logistic-regression algorithm, a decision-tree algorithm, a support vector machine (SVM) algorithm, a k-Nearest-Neighbors (kNN) algorithm, a gradient-boosting algorithm, a random-forest algorithm, or an artificial neural network (ANN), such as a four-dimensional convolutional neural network (CNN). For example, a gradient-boosting model may comprise a series of trees where each subsequent tree minimizes a predictive error of the preceding tree. Accordingly, in some examples in which condition estimator 264 uses a machine-learning model to determine a matching skin-condition type, the basis for the determination may be sufficiently encapsulated within the machine-learning model so as not be readily apparent (e.g., not clearly objectively articulable) to the user. Upon determining one or more matching skin-condition types for skin condition 102, condition estimator 264 is configured to transfer the determined skin-condition type(s) to development predictor 266.

In general, development predictor 266 is configured to predict, based at least in part on the determined skin-condition type, a unique future development of the patient's skin condition 102 over time. For example, development predictor 266 may receive the determined skin-condition types from condition estimator 264, and either a copy of 3-D polygon mask 320 from mesh builder 262, a copy of revised 2-D imagery 326 from condition estimator 264, or both.

Based on the determined skin-condition types, development predictor 266 determines (e.g., generates, receives, or retrieves from storage device(s) 208) a corresponding set of modeling data 220 for each determined skin-condition type. Modeling data 220 describes an average or “typical” developmental behavior of each skin-condition type. The typical developmental behavior may include, as non-limiting examples, a typical growth rate, a typical growth pattern, a typical growth direction, a typical change in relative severity, a typical change in coloration, typical growth regions on patients' bodies, a typical change in texture, or any other description of a known, statistically probable change in the respective skin-condition over time.

In some examples, modeling data 220 may include multiple different “typical” developmental datasets based on different variables. As one illustrative example, modeling data 220 may include, for a particular skin-condition type, a first dataset describing a typical development of the skin-condition type in the absence of medical treatment, and a second dataset describing a typical development of the skin-condition type in response to effective medical treatment, or any other similar developmental scenario based on controllable variables.

Development predictor 266 may then determine, based on the current parameter values of the patient's skin condition 102 (e.g., indicated by 3-D polygon mesh 320 and/or revised 2-D imagery 326), and based on the typical development of the determined skin-condition type (e.g., indicated by modeling data 220), a set of predicted future parameter values of the patient's skin condition at various points in time. In other words, polygons 324 (of 3-D polygon mask 320 and/or 2-D imagery 326) represent (e.g., encode) a set of initial conditions that are unique to patient 108. On the other hand, modeling data 220 represents (e.g., encodes) a most-probable rate-of-change for each skin-condition parameter as experienced by many prior patients. Conceptually, development predictor 266 is configured to apply the “rate of change” information (e.g., modeling data 220) to the “initial condition” information (e.g., polygons 324), in order to predict a unique future development of patient's 102.

In one specific example, development predictor 266 is configured to use modeling data 220 and polygons 324 to produce, for each descriptive skin-condition parameter of skin condition 102, a mathematical function that models a change in the parameter over time. Each mathematical function may be configured to receive, as an independent variable, a value representing a future point in time (e.g., a value of “2” representing two weeks into the future), and output, based on the independent variable, a corresponding predicted future value for the respective skin-condition parameter.

In some such examples, development predictor 266 may be configured to automatically generate, based on a set of stored, predetermined values for the independent time variable, a set of predicted future states of development of skin condition 102, wherein each future state of development includes a predicted future dataset of associated values for each skin-condition parameter at the respective predetermined point in time indicated by each predetermined time value. In some such examples, development predictor 266 may be configured to generate, for each predicted future dataset, a respective plurality of “future” polygons, wherein each set of future polygons graphically depicts a developmental stage of skin condition 102 in a way that satisfies the predicted future dataset.

In other examples, development predictor 266 includes a neural-network-based model trained to predict the future development of skin condition 102 based on polygons 324 and modeling data 220 as input. For example, development predictor 266 may apply a custom neural-network in order to graphically predict the developmental stages of skin condition 102, or in other words, to automatically generate and output each set of future polygons. Development predictor 266 may then transfer the mathematical developmental functions, the predicted future datasets, and/or the pluralities of future polygons, to model generator 268.

In general, model generator 268 is configured to generate a virtual 3-D developmental model that includes a plurality of predicted growth-stage models, each growth-stage model graphically depicting a predicted future development of skin condition 102 at a different point in time. As one example, model generator 268 is configured to receive the various plurality of predicted future polygons, and assemble each set of future polygons into a 3-D growth-stage model. For instance, while decomposing 3-D virtual mesh 320 into individual polygons 324, condition estimator 264 may have selected a “reference” polygon from among individual polygons 324, and then generated a reference dataset describing the relative and orientations of all of the other polygons 324 relative to the reference polygon. Accordingly, each set of future polygons may include a respective reference polygon that corresponds to the original reference polygon of polygons 324. Therefore, model generator 268 may be configured to use the reference dataset to re-align all of the other future polygons relative to the reference polygon of the respective set, thereby constructing a set of virtual 3-D growth-stage models, collectively making up a 3-D developmental model 330 (FIG. 3D) for skin condition 102. Model generator 268 may then transfer the 3-D developmental model 330, comprising the set of growth-stage models, to XR generator 270.

In general, XR generator 270 is configured to generate and output extended-reality (XR) content (e.g., XR imagery 112 of FIG. 1) that includes 3-D developmental model 330 or other graphical imagery derived therefrom. XR generator 270 may be configured to generate and output different types of XR content based on the particular type of XR device being used to display the content. As one example, when outputting content for display on a transparent visor of an MR headset, XR generator 270 may generate a 2-D projection of 3-D developmental model 330, and anchor the 2-D projection onto the visor relative to the location of the patient's affected area 104, as viewed from the perspective of a user wearing the MR headset. In other examples, when outputting content for display on a (non-transparent) display screen of a virtual-reality (VR) headset, XR generator 270 may generate a 2-D projection of 3-D developmental model 330 overlaid onto a virtual model of the patient's affected skin area 104, or a virtual avatar of patient 108. In other examples, such as the example illustrated in FIG. 3D, when outputting content to display screen 238 of mobile device 230, XR generator 270 may generate composite 2-D imagery 346 based on real-time 2-D images 332 and 3-D developmental model 330.

In accordance with techniques of this disclosure, XR generator 270 is configured to generate XR content through a distance-based object-rendering approach. For example, as illustrated and described with respect to FIG. 3D, XR generator 270 is configured to receive updated, current, or real-time 2-D imagery 332 of the patient's affected area 104 from camera 244. XR generator 270 then identifies feature points 322 within 2-D imagery 332, which may include some or all of the same feature points 322 identified by mesh builder 262, as described above. Based on the relative locations of feature points 322 within 2-D imagery 332 (e.g., the distances to one another and from the camera 244), XR generator 270 defines a virtual axis 334 within 2-D imagery 332.

XR generator 270 may continue to receive updated or real-time sensor data 226, such as IMU data and depth-sensor data. Based on updated sensor data 226, XR generator 270 determines and monitors the relative location and orientation of virtual axis 334, in order to determine and monitor a relative distance and orientation between the camera 224 and the patient's affected skin area 104, as depicted within current 2-D imagery 332. Based on virtual axis 334 and the monitored relative distance, XR generator 236 determines (e.g., selects or identifies) an augmentation surface 340 an area within 2-D imagery 332 on which to overlay virtual content, such as 3-D developmental model 330. In some examples, but not all examples, augmentation surface 340 includes the patient's affected skin area 104, which, as described above, may include the same feature points 322 previously identified by mesh builder 262.

Based on the monitored relative location and orientation of virtual axis 334 within current imagery 332, XR generator 270 determines a corresponding size and relative orientation at which to generate a 2-D projection of 3-D developmental model 330 (e.g., to align developmental model 330 with virtual axis 334). For example, if XR generator 270 determines that virtual axis 334 is getting “farther away” from camera 244, as indicated by current imagery 332, XR generator 270 generates a relatively smaller 2-D projection of 3-D developmental model 330, and conversely, a relatively smaller 2-D projection when virtual axis is nearer to camera 244.

XR generator 270 may then generate a composite image 346 by overlaying the 2-D projection of 3-D developmental model 330 onto augmentation surface 340 within current imagery 332. For example, XR generator 270 may identify corresponding (e.g., matching) feature points 322 within both of the current imagery 332 and the 2-D projection of 3-D developmental model 330, and overlay the 2-D projection onto current imagery 332 such that the corresponding pairs of feature points 322 overlap. In other words, XR generator 270 may position each growth-stage model by matching feature points 322 in the initial 2-D image 332 with the feature points 322 in the graphical texture of 3-D developmental model 330, and anchoring the 3-D developmental model 330 above the pre-rendered mesh of target augmentation surface 340. In some examples, XR generator may perform an iterative alignment process, by repeatedly adjusting the position of the 2-D projection relative to the 2-D image so as to reduce or minimize an error (e.g., a discrepancy) between corresponding matched feature points.

XR generator 270 then outputs composite image 346 to display screen 238 of mobile device 230. In this way, XR generator 270 (e.g., via a graphics processing unit (GPU) of mobile device 230), renders XR (e.g., AR) content and displays real-time AR developmental stages of skin condition 102 overtop of the patient's affected skin area 104.

In some examples, skin-condition modeler 224 is configured to identify and correct for anomalies or other errors, such as while estimating a skin-condition type, or while predicting and visualizing the future development of skin condition 102. For example, skin-condition modeler 224 may receive user input (e.g., feedback from a dermatologist or other user) indicating an anomaly, such as an incorrectly estimated skin-condition type or an implausible development (e.g., excessive or insufficient growth, change in coloration, or the like) within 3-D developmental model 330. As one example, a user may submit a manual correction for one or more of the individual growth-stage models of 3-D developmental model 330. In examples in which condition estimator 264 includes a machine-learned model trained to estimate the skin-condition type, and/or examples in which development predictor 266 includes a machine-learned model trained to generate the growth-stage models, upon receiving a manual correction or other user feedback, skin-condition modeler 224 may be configured to automatically perform batch-wise (e.g., complete) retraining of either or both of these skin-condition-predictive models, using the user's feedback as new training data. In some such examples, in which the “magnitude” of the user's correction (e.g., the magnitude of the difference between the user's indication of the “correct” developmental pattern and the automatically generated “incorrect” developmental pattern) exceeds a pre-determined threshold, skin-condition modeler 224 may be configured to generate and output a notification that the machine-learning model is operating outside acceptable variance limits, and that the model may need to be updated (as compared to merely retrained) by the developer.

FIG. 4 is a conceptual diagram depicting an example of the skin-condition-prediction system 100 of FIG. 1. More specifically, FIG. 4 depicts an example including computing system 400 that includes two computing devices: a “local” computing device 230 and a “remote” or “cloud” computing server 236. This example configuration is advantageous in that the cloud server 236 may include greater processing power, and therefore may be better-suited to handle the more-computationally-intensive, but less-time-sensitive techniques of a skin-condition-prediction process, such as estimating a skin-condition type. Conversely, the local device 230 can better-handle the less-resource-intensive, but more-time-sensitive techniques, such as updating an orientation of virtual objects in real-time. Computing system 400 may be an example of computing system 200 of FIG. 2A, in that each individual computing unit 230, 236 may include any or all of the example components of computing system 200.

Local device 230 includes virtually any suitable computing device that is physically (e.g., geographically) local to the user, such as a smartphone, laptop, a desktop computer, a tablet computer, a wearable computing device (e.g., a smartwatch, etc.), or the like. Local device 230 is configured to receive or capture, from various sensors 228, sensor data 226 including 2-D images 306 depicting a skin condition 102 on an affected area 104 of a body 106 of a patient 108 from multiple different perspectives. Other types of sensors may include a depth sensor (e.g., LIDAR and/or infrared-based depth sensing), and a 9-axis IMU 240. In some examples, local device 230 may be configured to wirelessly transfer the sensor data 226 to cloud computing system 236. In other examples, local device 230 retrains the sensor data 226 and performs any or all of the functionality of cloud-computing system 236 described below.

Cloud computing system 236 (also referred to herein as “CCS 236”) is configured to receive the sensor data 226, including the 2-D images from mobile device 230. CCS 236 compares the 2-D image(s), or other 2-D imagery derived therefrom, to stored models of skin conditions in order to determine which condition classification best matches the condition in the 2-D imagery. In some examples, CCS 236 feeds the 2-D imagery into a neural network (e.g., a convolutional neural network (CNN)) trained to estimate or identify a matching skin-condition type. In some such examples, CCS 236 may be configured to map each pixel of the 2-D imagery to a different input neuron in a 2-D array of input neurons in order to perform pixel-based pattern and texture recognition.

CCS 236 may then determine (e.g., retrieve, receive, generate, etc.) modeling data based on the determined skin-condition classification, and may generate a set of predicted growth-stage models of skin condition 102, e.g., characterizing, via colored texture data, a predicted direction of growth, a predicted coloration, a predicted relative severity, etc., of the skin condition 102. CCS 236 may then construct the growth-stage models over a 3-D curved polygon mesh (collectively forming a 3-D developmental model 330) and may send the 3-D mesh (along with the colored texture data of the growth-stage models) back to local device 230.

Local device 230 is configured to monitor (e.g., determine, at regular periodic intervals) a location and orientation of a virtual axis (e.g., axis 334 of FIG. 3D) and a relative distance between, for example, a camera or other sensor of local device 230 and the patient's body 108, in order to identify a “plane of augmentation” or augmentation surface 340, or in other words, a surface depicted within the 2-D image(s) on which to overlay virtual content. Local device 230 is further configured to anchor the 3-D developmental model 330 to the plane of augmentation based on a set of identified anchor points 322 along the plane of augmentation 340. Local device 230 is further configured to monitor the augmented surface area based on the monitored depth and movement data recorded by the integrated IMU 240, and to update the relative position of the 3-D developmental model accordingly. For example, local device 230 may be responsible for: (1) determining and capturing a relative distance of the patient's area of interest (e.g., affected skin area 104) from the local device 230; (2) determining and capturing movement subtleties based on IMU data; and (3) controlling display settings, such as adjusting a brightness, contrast, hue, and saturation according to lighting and environmental conditions. In this way, computing system 400 is configured to animate a predicted future progress of the skin condition 104

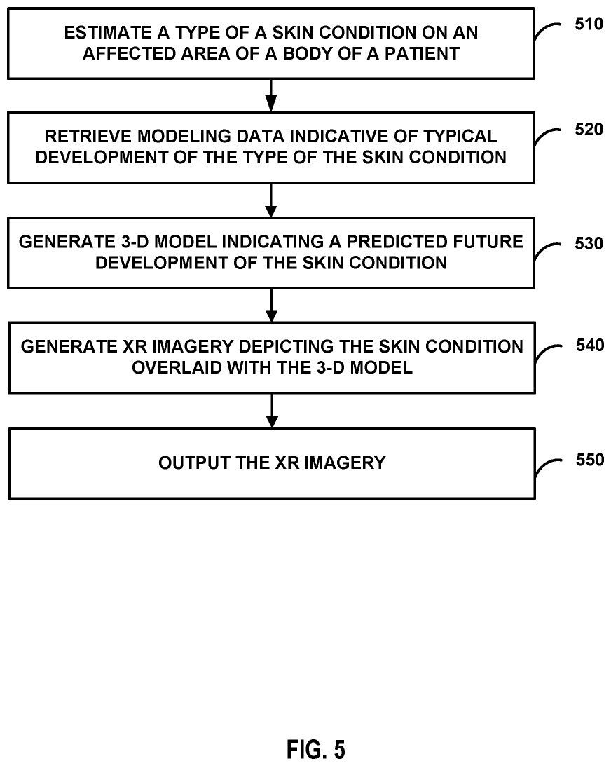

FIG. 5 is a flowchart illustrating an example skin-condition-prediction process, in accordance with one or more aspects of the techniques disclosed. The techniques of FIG. 5 are described primarily with respect to the example hardware architecture of computing system 200 of FIG. 2B and the example software modules of FIG. 2C, however, any suitable computing system may perform the techniques herein.

A computing system 200 having one or more processors is configured to estimate a skin-condition type or category for a skin condition 102 on an affected area 104 of a body 106 of a patient 108 (510). For example, the computing system may receive 2-D image data 306 depicting the affected skin area 104 from multiple perspectives, and then perform a 2-D-to-3-D-to-2-D image-conversion process in order to produce a graphical depiction of, for example, the size, shape, texture, pattern, and/or coloration of the skin condition 102. Computing system 200 may then identify one or more probable skin-condition types based on the 2-D image data and the stored skin-condition-type data 218 indicative of known types of skin conditions. For example, computing system 200 may perform the 2-D-to-3-D-to-2-D process described elsewhere in this disclosure and apply a machine-learning model to the refined 2D data to estimate the skin-condition type.

Based on the identified skin-condition type(s), computing system 200 may determine (e.g., retrieve or generate) modeling data 220 describing a typical developmental behavior for the estimated skin-condition type(s) (520). For example, the data may indicate a typical change in size, shape, coloration, texture, or relative severity, of the respective type of skin condition.

Based on the modeling data, computing system 200 generates a 3-D developmental model 330 indicating (e.g., graphically depicting) a predicted future development (e.g., at least a predicted direction of growth) of the patient's skin condition 102 (530). For example, computing system 200 may apply the refined 2-D data and the modeling data into a machine-learning model trained to generate a plurality of virtual growth-stage models indicating a development of the skin condition at different pre-determined points of time in the future.

The computing system 200 may use the 3-D developmental model 330 to generate extended-reality (XR) imagery or other XR content (540). For example, the computing system 200 may generate composite imagery 346 depicting the patient's affected skin area 104 overlaid with a 2-D projection of the 3-D developmental model 330. The computing system 200 may output the XR imagery 346 to a display device, such as a display screen 238 of a mobile device 230 (550). The computing system 200 may update the XR content in real-time based on a motion of the mobile device 230 relative to the affected skin area 104 (as indicated by an integrated IMU 240), in order to create the appearance of the 3-D developmental model 330 “anchored” to the patient's affected skin area 104.

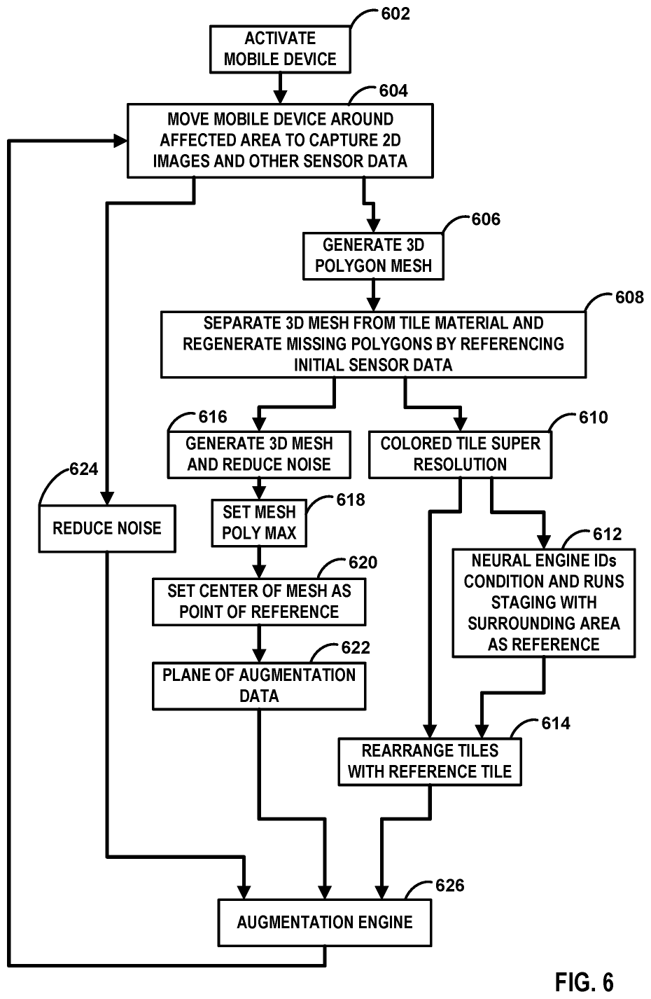

FIG. 6 is a flowchart illustrating an example dermatological-condition-prediction process, in accordance with one or more aspects of the techniques disclosed. As one non-limiting example, the techniques of this disclosure include a computing system configured to capture sensor data (including 2-D image data) for a patient's skin condition, feed the collected data through a deep-learning model configured to estimate the type of skin condition and predict its future development, and generate and output extended-reality imagery visualizing the predicted future development. The techniques of FIG. 6 are described primarily with respect to the example hardware architecture of computing system 200 of FIG. 2B, however, any suitable computing system may perform the techniques herein.

A user (e.g., a patient 108 or a clinician of patient 108) of a mobile device 230 activates a skin-condition-visualization application, such as skin-condition modeler 224 of FIG. 2A, running on mobile device 230 (602). While activated, mobile device 230 may be configured to actively stream data, such as sensor data 226 via data-streaming device(s) 232 of FIG. 2B. In other examples, mobile device 230 may be configured to periodically transfer data (e.g., sensor data 226) via data-streaming device(s) 232, or after after the data has been captured.

In response to a prompt, the user may select an “Automatic Capture” mode or a “Manual Capture” mode. Upon selecting the “Manual Capture” mode, the user may be further prompted to select a target area within a 2-D image 306 depicting a skin condition 102 on an affected skin area 104 on the body 106 of a patient 108. Upon selecting the “Automatic Capture” mode, skin-condition modeler 224 may attempt to automatically locate the skin condition 102 within the 2-D image 306.

In response to a prompt (e.g., appearing on display screen 238), the user may then move the mobile device 230 around the affected skin area 104 (604). While the mobile device is in motion, an integrated camera 244 captures 2-D images 306 of the affected skin area 104, while other integrated sensors 228, such as a 9-axis IMU 240 and a depth sensor 242, capture additional sensor data 226 describing the relative position, orientation, and/or motion of mobile device 230 at any given point in time.

Using the 2-D images 306 and the other sensor data 226, skin-condition modeler 224 generates a 3-D polygon mesh 320 (606), such as a curved 3-D surface made up of a plurality of 2-D polygons 324 (so as to mimic the curvature 304 of the patient's body) overlaid with a graphical texture representing the affected area 104 of the patient's body.

Skin-condition modeler 224 may then make a copy of 3-D polygon mesh 320 and deconstruct the mesh 320 into the individual 2-D polygons 324. For example, skin-condition modeler 224 may “separate” the 3-D mesh 320 from the 2-D polygons or “tiles” 324 that make up the outer surface of the 3-D mesh (608). Skin-condition modeler 224 may then flatten the tiles 324 onto a common 2-D plane, and fill in any gaps between adjacent tiles, thereby producing revised 2-D imagery 326 depicting the size, shape, color, texture, and pattern of the patient's skin condition 102. In some examples, but not all examples, skin-condition modeler may be configured to apply revised 2-D imagery 326 to a “super-resolution” neural network, trained to increase the resolution of 2-D imagery 326 even further (e.g., by extrapolating particularly high-resolution patterns and textures into lower-resolution areas, smoothing pixel edges, etc.) (610).

In some examples, skin-condition modeler 224 may prompt the user to input or select a type, category, or label for skin condition 102, if known to the user. In other examples, an AI or deep-learning model, such as a neural engine, analyzes the color, texture, and pattern within the revised 2-D imagery 326 in order to “identify” a type or category to which skin condition 102 most-likely belongs (612). Based on a typical developmental behavior of the identified type of skin condition, the neural engine predicts a unique (e.g., patient-specific) future development of skin condition 102. For example, the neural engine may use the surrounding affected skin area 104 (as depicted on tiles 324) as a reference, e.g., a starting point or set of initial conditions, to apply to the typical developmental behavior in order to generate a plurality of virtual growth-stage models depicting the predicted future development of skin condition 102.

In examples in which the virtual growth-stage models each includes a respective 2-D image based on revised 2-D imagery 326 (e.g., based on individual tiles 324), the neural engine may then convert the virtual growth-stage models into curved 3-D growth-stage models by rearranging (e.g., reassembling) individual tiles relative to a designated reference tile (614).

Skin-condition modeler 224 generates a subsequent 3-D mesh (which may substantially conform to the shape and/or structure of the original 3-D mesh), and reduces noise in the 3-D mesh, such as by averaging-out above-threshold variations in the curvature of the surface of the subsequent 3-D mesh (616). In some examples, skin-condition modeler 224 may “smooth” the 3-D mesh into a curved surface by first determining (e.g., extrapolating) a curvature of the 3-D mesh, and then simultaneously increasing the number and reducing the size of the individual polygons making up the 3-D mesh, thereby increasing the “resolution” of the 3-D mesh in order to better-approximate the appearance of a smooth curve (618).

Skin-condition modeler 224 may identify a centerpoint of the subsequent 3-D mesh and designate the centerpoint as a point of reference (620). For example, skin-condition modeler 224 may define a virtual axis 334 passing through the centerpoint, and use the axis 334 as a basis for orientation and alignment of 3-D mesh 330 relative to subsequent 2-D imagery 332.

Skin-condition modeler 224 may identify, based on virtual axis 334 and subsequent 2-D imagery 332 captured by camera 244, a plane of augmentation 340, or in other words, a “surface” depicted within the 2-D images 332 upon which virtual objects will be shown or overlaid (622).

Skin-condition modeler 224 may reduce an amount of noise (e.g., average-out excessive variation) within sensor data 226 (624), and then feed sensor data 226, the subsequent 3-D mesh, the subsequent 2-D imagery 332, the augmentation plane 340, and the virtual growth stage models into an augmentation engine (e.g., XR generator 270 of FIG. 2C) configured to generate and output XR content 346 (626). For example, the XR content may include composite imagery depicting the patient's affected skin area 104 overlaid with a 2-D projection of 3-D developmental model 330, thereby modeling a predicted future progression of the skin condition 102 over time. Skin-condition modeler 224 may perform this dermatological-condition-prediction process in real-time, such that skin-condition modeler 224 may continue to generate and output this type of XR content in this way as the user continues to move camera 244 of mobile device 230 around the affected skin area 104 (604).

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units or engines is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method comprising:
estimating, by a computing system based on sensor data, a skin-condition type for a skin condition on an affected area of a body of a patient;
determining, by the computing system based on the sensor data and the predicted skin-condition type, modeling data indicative of a typical development of the skin-condition type;
generating, by the computing system based on the sensor data and the modeling data, a 3-dimensional (3-D) model indicative of a predicted future development of the skin condition over time;
generating, by the computing system, extended reality (XR) imagery of the affected area of the body of the patient overlaid with the 3-D model; and
outputting the XR imagery.

2. The method of claim 1, wherein the sensor data comprises 2-D imagery of the affected area of the body of the patient from a plurality of perspectives.

3. The method of claim 1, wherein the sensor data comprises:
2-D image data;
inertial measurement unit (IMU) data; and
depth-sensor data.

4. The method of claim 3, wherein generating the XR imagery comprises:
defining, by the computing system based on the sensor data, a virtual axis positioned relative to the affected area of the body of the patient;
monitoring, by the computing system based on the virtual axis, a relative distance between the affected area of the body of the patient and a sensor configured to generate the sensor data;
determining, by the computing system based on the virtual axis and the relative distance, an augmentation surface positioned relative to the affected area of the body of the patient within the 2-D image data; and
overlaying, by the computing system, the 3-D model onto the augmentation surface relative to the affected area within the 2-D image data.

5. The method of claim 4, wherein the augmentation surface comprises a plurality of feature points on the skin of the patient within the 2-D image data, and wherein generating the XR imagery comprises anchoring, by the computing device, the 3-D model to the augmentation surface based on the plurality of feature points.

6. The method of claim 1, further comprising calculating, based on the sensor data, a curved geometry of the affected area of the body of the patient, wherein generating the 3-D model comprises generating, by the computing system based on the curved geometry of the affected area, a curved polygon mask conforming to the curved geometry of the affected area.

7. The method of claim 1, wherein generating the 3-D model comprises:
determining, by the computing system based on the sensor data and the modeling data, at least a predicted direction of growth of the skin condition;
generating, by the computing system based on the predicted direction of growth, a plurality of growth-stage models for the skin condition; and
generating, by the computing system, the 3-D model comprising the plurality of growth-stage models.

8. The method of claim 1, wherein outputting the XR imagery comprises outputting for display, by the computing system, the XR imagery onto a display screen of a mobile computing device

9. The method of claim 1, wherein the 3-D model indicates a predicted direction of growth of the skin condition, a predicted relative severity of the skin condition, or a predicted coloration of the skin condition.

10. The method of claim 1, further comprising identifying, by the computing system, the affected area of the body of the patient by performing texture-and-color analysis on image data of the sensor data to locate the skin condition within the image data.

11. The method of claim 1, further comprising identifying, by the computing system, the affected area of the body of the patient based on user input indicative of a location within image data of the sensor data.

12. The method of claim 1, further comprising:
determining, by the computing system, an anomaly between the 3-D model and the skin condition of the patient; and
performing, by the computing system, batch-wise retraining of skin-condition-predictive models of the computing system.

13. The method of claim 1, wherein the computing system comprises a smartphone, a laptop, computer, a tablet computer, a wearable computing device, or an XR headset.

14. The method of claim 1, wherein the sensor data comprises 2-D image data, and wherein predicting the skin-condition type comprises:
converting, by the computing system, the 2-D image data into a 3-D polygon mesh;
converting, by the computing system, the 3-D polygon mesh into revised 2-D imagery; and
determining, by the computing system, the skin-condition type that corresponds to the skin condition in the revised 2-D imagery.

15. The method of claim 14, wherein determining the skin-condition type that corresponds the skin condition in the revised 2-D imagery comprises applying a machine-learned model to the revised-2-D imagery, wherein the machine-learned model is trained to determine the skin-condition type.

16. A computing system comprising processing circuitry configured to:
estimate, based on sensor data, a skin-condition type for a skin condition on an affected area of a body of a patient;
determine, based on the sensor data and the estimated skin-condition type, modeling data indicative of a typical development of the skin-condition type;
generate, based on the sensor data and the modeling data, a 3-dimensional (3-D) model indicative of a predicted future development of the skin condition over time;
generate extended reality (XR) imagery of the affected area of the body of the patient overlaid with the 3-D model; and
output the XR imagery.

17. The computing system of claim 16, wherein the processing circuitry is further configured to calculate, based on the sensor data, a curved geometry of the affected area of the body of the patient, wherein generating the 3-D model comprises generating, based on the curved geometry of the affected area, a curved polygon mask conforming to the curved geometry of the affected area.

18. The computing system of claim 16, wherein the sensor data comprises 2-D image data, and wherein predicting the type of the skin condition comprises:
converting the 2-D image data into a 3-D polygon mesh;
converting the 3-D polygon mesh into revised 2-D imagery; and
determining the skin-condition type that corresponds to the skin condition in the revised 2-D imagery.

19. The computing system of claim 18, wherein determining the skin-condition type that corresponds to the skin condition in the revised 2-D imagery comprises applying a machine-learned model to the revised 2-D imagery, wherein the machine-learned model is trained to determine the skin-condition type.

20. A non-transitory computer-readable storage medium having instructions stored thereon that, when executed, configure a computing system to:
estimate, based on sensor data, a skin-condition type for a skin condition on an affected area of a body of a patient;
determine, based on the sensor data and the estimated skin-condition type, modeling data indicative of a typical development of the skin-condition type;
generate, based on the sensor data and the modeling data, a 3-dimensional (3-D) model indicative of a predicted future development of the skin condition over time;
generate extended reality (XR) imagery of the affected area of the body of the patient overlaid with the 3-D model; and
output the XR imagery.
